(19) United States
(12) Patent Application Publication
(10) Pub. No.: US 2012/0100117 A1
(43) Pub. Date: Apr. 26, 2012

(54) METHOD FOR CHONDROGENIC DIFFERENTIATION OF PLURIPOTENT OR MULTIPOTENT STEM CELLS USING WNT6

(76) Inventors: Daniele Noel, Montpellier (FR); Claire Bony-Garayt, Montpellier (FR); Christian Jorgensen, Montpellier (FR)

(21) Appl. No.: 13/265,956

(22) PCT Filed: Apr. 24, 2009
(86) PCT No.: PCT/IB2009/052800

§ 371 Date: Jan. 4, 2012

(51) Int. Cl. A61K 35/12; A61P 19/02; A61P 19/00; C12N 5/077; C12N 5/10

(52) U.S. Cl. 424/93.21; 435/377; 435/325

(57) ABSTRACT

The present invention relates to methods for obtaining chondrocytes by culturing pluripotent or multipotent stem cells with a culture medium comprising Wnt6 or a derivative thereof. The inventive methods have the advantage of specifically leading to chondrogenic lineage and providing chondrocytes without differentiation towards hypertrophic chondrocytes, adipocytes or osteoblasts.

### FIELD OF THE INVENTION

The invention relates to a method for obtaining a population of chondrocytes, said method comprising a step of culturing pluripotent or multipotent stem cells with a culture medium comprising Wnt6 or a derivative thereof. The invention also relates to the use of Wnt6 or a derivative thereof for the treatment of osteo-articular pathologies, and/or for tissue reconstitution or regeneration, and/or the inhibition of the differentiation of mature chondrocytes to hypertrophic chondrocytes.

### BACKGROUND OF THE INVENTION

Healthy cartilage is a highly robust tissue, and is resilient against the stringent mechanical and biological constraints imposed upon it. Cartilage defects are common features of joint diseases, but current treatments can rarely restore the full function of native cartilage. Recent studies have provided new perspectives for cartilage engineering using pluripotent or multipotent stem cells. Indeed, because of their combined abilities of unlimited expansion and pluripotency, pluripotent or multipotent stem cells remain a potential source for regenerative medicine and tissue replacement after injury or disease.

For example, multipotent mesenchymal stromal cells are progenitor cells mainly isolated from bone marrow or fat tissue with the capacity to differentiate into multiple lineages and in particular, to bone, adipose tissue and cartilage (Dominici et al., 2006). These cells represent an attractive cell source for tissue engineering for skeletal disorders when regeneration is affected. This is the case of cartilage which is a tissue with poor or no capacity of self-regeneration. Such limitated capacity of cartilage to regenerate represents a major obstacle in the management of degenerative and traumatic injuries.

Chondrogenesis is a process involving stem-cell differentiation through the coordinated effects of growth/differentiation factors and extracellular matrix (ECM) components. It results consequently that one major goal for such clinical applications based on stem cell-based therapies consists in providing a chondrogenic factor suitable for specifically triggering differentiation of these pluripotent or multipotent stem cells towards the desired lineage and maintenance of the differentiated phenotype, namely the chondrogenic lineage.

On the other hand, the Wingless (Wnt) family of secreted ligands contains more than 20 members in vertebrates that are characterized by conserved cysteine residues. These proteins exhibit unique expression patterns and distinct functions in development (Kikuchi et al., 2007). Wnts bind Frizzled (Fz) proteins which are seven-pass transmembrane receptors with an extracellular N-terminal cysteine-rich domain (CRD). A single Wnt can bind multiple Fzd proteins and vice-versa (Clevers et al., 2006). In addition, Fzds cooperate with single-pass transmembrane molecules of the LRP family: LRP5 and LRP6. The Wnt family members can be divided into two groups based upon their ability to induce or not the canonical β-catenin-dependent pathway. The canonical pathway is highly conserved among various species. In the absence of Wnt, the serine-threonine kinases, casein kinase 1α (CK1α) and glycogen-synthase kinase-3β (GSK-3β), phosphorylate β-catenin in the Axin complex. As a result, phosphorylated β-catenin is ubiquitinated and degraded by the proteasome (Gordon et al., 2006). Once bound by Wnts, Fzd in the Fzd/LRP coreceptor complex phosphorylates Dishevelled (Dsh) and Axin is recruited to LRP upon phosphorylation of LRP by CK1α and GSK-3β. The recruitment of Axin away from the destruction complex leads to the stabilization of β-catenin. The accumulated β-catenin translocates into the nucleus, where it binds to the transcription factor T cell factor (Tcf)/lymphoid enhancer factor (Lef) and thereby stimulates the expression of various target genes. Some Wnts activate a β-catenin-independent pathway. This activation occurs via at least three mechanisms in vertebrates. Some Wnts can increase the intracellular Ca²⁺ concentration and activate the calcium/calmodulin-dependent protein kinase II and protein kinase C (PKC) (Kikuchi et al., 2007). Other Wnt/Fzd interactions lead to the activation of phospholipase C and phosphodiesterase. Finally, Fzd can act through the activation of small G proteins, including Rac and Rho, c-jun N-terminal kinase (JNK) and Rho-associated kinase.

Various Wnt members are involved both in early and late skeletal development and play a role in the control of chondrogenesis and hypertrophy. Wnt1, Wnt4, Wnt7a, Wnt8 block chondrogenic differentiation but display different effects on hypertrophy. On the contrary, Wnt3a, Wnt5a and Wnt5b promote early chondrogenesis (Church et al., 2002) and Wnt11 does not affect chondrogenic differentiation. Wnt members responsible for the induction of the osteogenic differentiation were shown to activate the β-catenin-dependent pathway, while repressing the Sox9 chondrogenic transcription factor in fetal development (Hill et al., 2005; Spater et al., 2006). However, β-catenin was recently shown to be required for both osteogenesis and chondrogenesis in adult mature tissues (Chen et al., 2007).

Murine Wnt6 was cloned from developing fetus based on homology sequences to Wnt1 (Gavin et al., 1990). In the embryo, the precise sites of Wnt6 expression coincide with zones of epithelial remodelling and epithelial-mesenchymal transformation and are closely associated with areas of BMP signalling (Schubert et al., 2002). Wnt6 regulates the mesenchymal to epithelial transition of the segmental mesoderm leading to somite formation. Somites consist of an epithelial ball enclosing mesenchymal cells taking part in the formation of intervertebral disks and joints (Schmidt et al., 2004). Wnt6 was required for the maintenance of the epithelial structure of somites through binding to Fzd7 receptor, activation of the β-catenin-dependent pathway and targeting the paraxis transcription factor (Linker et al., 2005). However, in another model of C57MG mammary epithelial cells, Wnt6 failed to induce the canonical Wnt pathway and led to a weak transformation of the cells (Shimizu et al., 1997). However, no investigation on the role of Wnt6 has been made on the chondrogenic differentiation of pluripotent or multipotent stem cells.

Moreover, currently used chondrogenic factors such as BMP-2 or TGFβ3 do not induce a specific chondrogenic differentiation of pluripotent or multipotent stem cells such as MSCs since these factors also induce an osteogenic differentiation of these cells.

Besides, in joint pathology including osteoarthritis (OA), chondromalacia, hemochromatosis, cartilage traumatic defect, cartilage injury in chronic inflammatory arthritis, bone formation is characterised by calcification of adult cartilage by hypertrophic chondrocytes. Indeed, mature chondrocytes may sometimes continue their differentiation by the formation of hypertrophic chondrocytes (i.e. terminal differentiation of chondrocytes). For example, such phenomenon occurs with degenerative joint pathology including chondrocalcinosis, or in osteoarthritic patients.

Therefore, there is still a need in the art for a method of obtaining specifically a population of chondrocytes from pluripotent or multipotent stem cells without leading to the formation of hypertrophic chondrocytes and/or providing other lineages such osteogenic or adipogenic lineages.

### SUMMARY OF THE INVENTION

### Detailed Description of the Invention

The inventors have demonstrated the key role of Wnt6 in chondrogenesis and more particularly in the induction of this process through the activation of the non canonical β-catenin-independent Wnt pathway. Indeed, in attempt at identifying a suitable chondrogenic agent, the inventors have detected an early and transitory up-regulation of Wnt6 and more importantly they have shown that the addition of conditioned medium comprising the Wnt6 polypeptide allows the differentiation of pluripotent or multipotent stem cells specifically towards the chondrocytic lineage. Furthermore, Wnt6 ensure not only a suitable and specific signal for triggering differentiation of pluripotent or multipotent stem cells such MSCs towards the chondrogenic lineage but also the maintenance of the differentiated phenotype. Indeed, the inventors have also shown that Wnt6 inhibits the differentiation of mature chondrocytes to hypertrophic chondrocytes.

### DEFINITIONS

The term “pluripotent stem cells” as used herein refers to undifferentiated cells which have the potential to differentiate into any of the three germ layers: endoderm (interior stomach lining, gastrointestinal tract, the lungs), mesoderm (muscle, bone, blood, urogenital), or ectoderm (epidermal tissues and nervous system). Pluripotent stem cells can thus give rise to any fetal or adult cell type. However, alone they cannot develop into a fetal or adult animal because they lack the potential to contribute to extraembryonic tissue, such as the placenta. Typically, pluripotent stem cells may express the following markers Oct4, Sox2, Nanog, SSEA 3 and 4, TRA 1/81, see International Stem Cell Initiative recommendations, 2007.

As used herein, the term “embryonic stem cells” or “ES cells” or “ESC” refers to precursor cells that are pluripotent and have the ability to form any adult cell. ES cells are derived from fertilized embryos that are less than one week old. For example, human embryonic stem cells may be obtained according a protocol not involving the embryo destruction as described in Chung et al. 2008 or in Revazova et al. 2008.

As used herein, the term “induced pluripotent stem cells” or “iPS cells” or “iPSCs” refers to a type of pluripotent stem cell artificially derived from a non-pluripotent cell (e.g. an adult somatic cell). Induced pluripotent stem cells are identical to embryonic stem cells in the ability to form any adult cell, but are not derived from an embryo. Typically, an induced pluripotent stem cell may be obtained through the induced expression of Oct3/4, Sox2, Klf4, and c-Myc genes in any adult somatic cell (e.g. fibroblast).

For example, human induced pluripotent stem cells may be obtained according to the protocol as described by Takahashi K. et al. (2007), by Yu et al. (2007) or else by any other protocol in which one or the other agents used for reprogramming cells in these original protocols are replaced by any gene or protein acting on or transferred to the somatic cells at the origin of the iPS lines. Basically, adult somatic cells are transfected with viral vectors, such as retroviruses, which comprises Oct3/4, Sox2, Klf4, and c-Myc genes.

The term “multipotent stem cells” as used herein refers to a stem cell that has the potential to give rise to cells from multiple, but a limited number of lineages.

For example, adult human multipotent stem cells that can be used in the methods of the present invention include but are not limited to, multipotent mesenchymal stromal cells (MSCs), adult multilineage inducible (MIAMI) cells (D'Ippolito G et al., 2004), MAPC (also known as MPC) (Reyes M et al., 2002), cord blood derived stem cells (Kogler G et al., 2004), and mesoangioblasts (Sampaolesi M et al., 2006; Dellavalle A et al., 2007).

As used herein, the terms “multipotent mesenchymal stromal cells”, “mesenchymal stem cells” or “MSCs” are used herein interchangeably and refer to cells which are isolated mainly from bone marrow and adipose tissue (or fat tissue) but which have also been identified in other tissues such as synovium, periosteum or placenta. These cells are characterised by their property to adhere to plastic, their phenotype and their ability to differentiate into three different lineages (chondrocytes, osteoblasts and adipocytes). It must be noted that MSCs do not express Wnt6.

As used herein, the term “chondrocytes” refers to cells that are capable of expressing characteristic biochemical markers, including but not limited to collagen type II, notably type IIB, aggregan and also Sox9, cartilage oligomeric protein (COMP), link protein, and secreting a proteoglycan-rich extracellular matrix which can be shown by histological staining such as safranin O or alcian blue staining.

As used herein, the term “hypertrophic chondrocytes” refers to cells to the terminal step of the differentiation of the chondrocytic lineage that are capable of expressing characteristic biochemical markers, including but not limited to collagen 10, MMP13 and alkaline phosphatase.

As used herein, the term “culture medium” refers to any medium capable of supporting the growth and the differentiation of pluripotent stem cells or multipotent stem cells such as MSCs into chondrocytes in tissue culture. Media formulations that will support the growth and the differentiation of pluripotent stem cells or multipotent stem cells into chondrocytes include, but are not limited to, Dulbecco's Modified Eagle's Medium (DMEM). Typically, 0 to 10% Fetal Calf Serum (FCS) and basic fibroblast growth facto (bFGF) will be added to the above media in order to support the growth of pluripotent stem cells or multipotent stem cells and chondrocytes. However, a defined medium may be used if the necessary growth factors, cytokines and hormones in FCS for multipotent pluripotent stem cells or multipotent stem cells and chondrocytes identified and provided at appropriate concentrations in the growth medium. Antibiotics that can be supplemented into the culture medium include, but are not limited to, penicillin and streptomycin. A defined medium consisting of DMEM supplemented with 1% ITS (insulin, transferrin and selenium) solution, acid ascorbic 2-phosphate, proline and sodium pyruvate may be typically used as incomplete chondrogenic medium. A glucocorticoid can also be added in the culture medium such as dexamethasone.

As used herein, the terms “Wnt6” and “Wnt6 polypeptide” are used herein interchangeably and refer to the polypeptide sequence of the Wnt6 protein. The naturally occurring murine protein has an aminoacid sequence shown in Genbank, Accession number AAH48700 and the naturally occurring human protein has an aminoacid sequence shown in Genbank, Accession number AAG45154. The term “Wnt6” is used indifferently to designate recombinant, synthetic or native (i.e. isolated endogenous) Wnt6 protein. In the context of the invention, “Wnt6” further includes derivatives thereof such as fragments or variants of Wnt6, in particular of human Wnt6. For instance, derivatives may include all the Wnt6 protein produced by other species, e.g. rat, murine (SEQ ID NO: 1).

As used herein, a “Wnt6 derivative thereof” encompasses Wnt6 variants and Wnt6 fragments.

As used herein, a “Wnt6 variant” encompasses polypeptides having at least about 80 percent, or at least about 85, 90, 95, 97 or 99 percent sequence identity with the sequence of human Wnt6 (SEQ ID NO: 2). As used herein, “percentage of identity” between two amino acids sequences, means the percentage of identical amino-acids, between the two sequences to be compared, obtained with the best alignment of said sequences, this percentage being purely statistical and the differences between these two sequences being randomly spread over the amino acids sequences. As used herein, “best alignment” or “optimal alignment”, means the alignment for which the determined percentage of identity (see below) is the highest. Sequences comparison between two amino acids sequences are usually realized by comparing these sequences that have been previously align according to the best alignment; this comparison is realized on segments of comparison in order to identify and compared the local regions of similarity. The best sequences alignment to perform comparison can be realized, beside by using for example computer softwares using such algorithms (GAP, BESTFIT, BLAST P, BLAST N, FASTA, TFASTA). To get the best local alignment, one can preferably used BLAST software, with the BLOSUM 62 matrix, or the PAM 30 matrix. The identity percentage between two sequences of amino acids is determined by comparing these two sequences optimally aligned, the amino acids sequences being able to comprise additions or deletions in respect to the reference sequence in order to get the optimal alignment between these two sequences. The percentage of identity is calculated by determining the number of identical position between these two sequences, and dividing this number by the total number of compared positions, and by multiplying the result obtained by 100 to get the percentage of identity between these two sequences. It will also be understood that natural amino acids may be replaced by chemically modified amino acids. Typically, such chemically modified amino acids enable to increase the polypeptide half life.

A nucleic acid sequence “encoding” Wnt6 may have the coding sequence which, when translated, produces a protein having the same amino acid sequence as set forth in SEQ ID NO: 2 or a derivative thereof.

As used herein, a “Wnt6 fragment” is a biologically active portion of Wnt6 polypeptide. A “biologically active” portion of Wnt6 polypeptide includes a Wnt6-derived peptide that possesses one or more of biological activities of Wnt6.

Methods for producing recombinant proteins are known in the art. The skilled person can readily, from the knowledge of a given protein's sequence or of the nucleotide sequence encoding said protein, produce said protein using standard molecular biology and biochemistry techniques.

### Methods for Obtaining Chondrocytes

In a first aspect, the present invention relates to a method for obtaining a population of chondrocytes, said method comprising a step of culturing pluripotent or multipotent stem cells with a culture medium comprising Wnt6 or a derivative thereof.

In one embodiment, the pluripotent stem cells are human pluripotent stem cells.

In another embodiment, the pluripotent stem cells are non-human mammalian pluripotent stem cells.

Typically, said stem cells are embryonic stem cells. Thus, in a one embodiment, the pluripotent cells are human embryonic stem cells (hES cells) which are obtained according a method not involving the embryo destruction.

In another embodiment, the pluripotent stem cells are non-human embryonic stem cells, such a mouse embryonic stem cells.

In one embodiment, the pluripotent stem cells are induced pluripotent stem cells (iPS).

In another embodiment, the multipotent stem cells are human or non-human mammalian multipotent stem cells.

In a preferred embodiment, the human multipotent stem cells are multipotent mesenchymal stromal cells (MSCs).

According to this embodiment, MSCs are for example isolated from the bone marrow or the adipose tissue (adult origin) or isolated from the placenta (placental origin).

In one preferred embodiment, the obtained chondrocytes are mature chondrocytes.

In one embodiment, Wnt6 or a derivative thereof is directly added to the culture medium under the form of a polypeptide.

In a preferred embodiment, Wnt6 amino acid sequence is a mammal amino sequence, more preferably the murine amino sequence (SEQ ID NO: 1) and even more preferably the human amino sequence (SEQ ID NO: 2).

In another embodiment, Wnt6 is added to the culture medium under the form of a nucleic acid molecule.

Typically, said nucleic acid molecule is a DNA or RNA molecule, which may be included in any suitable vector, such as a plasmid, cosmid, episome, artificial chromosome, phage or a viral vector. The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence.

In other embodiments, a vector comprising a nucleic acid of the invention may be added to the culture medium.

Such vectors may comprise regulatory elements, such as a promoter, enhancer, terminator and the like, to cause or direct expression of said polypeptide upon administration to a subject. The vectors may further comprise one or several origins of replication and/or selectable markers. The promoter region may be homologous or heterologous with respect to the coding sequence, and provide for ubiquitous, constitutive, regulated and/or tissue specific expression, in any appropriate host cell, including for in vivo use. Examples of promoters include bacterial promoters (T7, pTAC, Trp promoter, etc.), viral promoters (LTR, TK, CMV-IE, etc.), mammalian gene promoters (albumin, PGK, etc.), and the like. Examples of plasmids include replicating plasmids comprising an origin of replication, or integrative plasmids, such as pUC, pcDNA, pBR, and the like. Examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+cells, 293 cells, etc. Protocols for producing such replication-defective recombinant viruses may be found for instance in WO 95/14785, WO 96/22378, U.S. Pat. No. 5,882,877, U.S. Pat. No. 6,013,516, U.S. Pat. No. 4,861,719, U.S. Pat. No. 5,278,056 and WO 94/19478.

In other embodiments, Wnt6 is produced in the culture medium by a host cell such as fibroblasts which have been transfected, infected or transformed by a nucleic acid and/or a vector according to the invention for differentiating pluripotent or multipotent stem cells into chondrocytes. The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed”.

In one embodiment, the host cells are pluripotent or multipotent stem cells so that they produced Wnt6.

Thus, the present invention relates also to a population of pluripotent or multipotent stem cells such as MSCs which have been transformed with a nucleic acid molecule encoding for Wnt6 or a vector comprising such nucleic acid.

In one preferred embodiment, MSCs are preferably isolated from the bone marrow or the adipose tissue of the patient into which the differentiated chondrocytes are to be introduced or transplanted.

The step of culturing pluripotent or multipotent stem cells with the culture medium comprising Wnt6 shall be carried out for the necessary time required for the production of chondrocytes. Typically, the culture of chondrocytes with the medium of the invention shall be carried out for at least 4 days, preferably at least 7 days, even more preferably at least 21 days. If necessary, the culture medium of the invention can be renewed, partly or totally, at regular intervals. Typically, the culture medium of the invention can be replaced with fresh culture medium of the invention every other day.

In one embodiment, pluripotent or multipotent stem cells are previously condensed together, for example, by centrifugation or by inclusion in a scaffold.

Thus, it is preferred that the pluripotent or multipotent stem cells are previously condensed together in order to form a micropellet culture system.

In one embodiment, the multipotent stem cells (e.g. MSCs) are preferably isolated from the bone marrow or the adipose tissue of a mammal, including humans.

In one preferred embodiment, the multipotent stem cells (e.g. MSCs) are obtained from the patient into whom the differentiated chondrocytes are to be introduced or transplanted.

In another embodiment, pluripotent or multipotent stem cells may be exposed to Wnt6 which is immobilized on a solid phase. According to the present invention, said solid phase is for example a plastic surface such as the plastic surface of culture wells or plates or a scaffold such as collagen- or hyaluronic acid-based scaffold. According to this embodiment, the pluripotent or multipotent stem cells are exposed to a Wnt6 immobilized on a solid phase, for at least 4 days, preferably at least 7 days, even more preferably at least 21 days.

The immobilization of Wnt6 may be direct or includes for example the use of a specific antibody directed to Wnt6 or another chemical compound which can bind Wnt6. The antibody or the chemical compound is then adsorbed on the solid phase.

In still another embodiment, the culture medium may further comprise at least another chondrogenic factor, which is selected from the group consisting for example of FGF-2, FGF-5, FGF18, IGF-1, TGF-beta1-3, BMP-2, BMP-7, Shh, Sox9, PDGF and VEGF.

In another aspect, the present invention relates to a population of chondrocytes obtainable by a method as defined above.

Advantageously, the population of chondrocytes according to the invention is homogenous, i.e. it is not necessary to perform any sorting or selection to isolate the cartilage precursors from other contaminating cells.

### Therapeutic Methods and Uses

In another aspect, the present invention relates to Wnt6 or a derivative thereof for regenerating cartilage and/or treating an osteo-articular pathology.

In another aspect, the present invention relates to a method for treating and/or preventing an osteo-articular pathology in a subject comprising a step of administrating a therapeutically effective amount of Wnt6 or a derivative thereof to a subject in need thereof.

In certain embodiments, a nucleic acid molecule encoding for thereof or a vector comprising such nucleic acid are used in regenerating cartilage and/or treating an osteo-articular pathology.

Yet another aspect of the invention relates to a population of chondrocytes of the invention or a population of pluripotent or multipotent stem cells which have been transformed as described above, for use in regenerating cartilage and/or treating an osteo-articular pathology. These cells are introduced into the surgery site to repair cartilage.

In one preferred embodiment, the multipotent stem cells are MSCs.

The invention also relates to a method for treating an osteo-articular pathology comprising the step of administering a pharmaceutically effective amount of a population of chondrocytes of the invention or a population of pluripotent or multipotent stem cells which have been transformed as described above to a patient in need thereof.

In one embodiment, the osteo-articular pathology is selected from the group consisting of degenerative cartilage lesions including osteoarthritis form knee or hip or any other joint and traumatic cartilage injuries such full-thickness, partial-thickness articular cartilage defects as well as consequences of chronic inflammation as in Rheumatoid Arthritis (RA) or Ankylosing arthritis (AS) or traumatic pathologies.

In this context, the terms “treating” or “treatment”, as used herein, refer to a method that is aimed at delaying or preventing the onset of a pathology, at reversing, alleviating, inhibiting, slowing down or stopping the progression, aggravation or deterioration of the symptoms of the pathology, at bringing about ameliorations of the symptoms of the pathology, and/or at curing the pathology.

As used herein, the term “subject” denotes a mammal, such as a rodent, a feline, a canine, and a primate. Preferably, a subject is a human.

As used herein, the term “pharmaceutically effective amount” refers to any amount of Wnt6 that is sufficient to achieve the intended purpose.

Effective dosages and administration regimens can be readily determined by good medical practice based on the nature of the pathology of the subject, and will depend on a number of factors including, but not limited to, the extent of the symptoms of the pathology and extent of damage or degeneration of the tissue or organ of interest, and characteristics of the subject (e.g., age, body weight, gender, general health, and the like). The dose and the number of administrations can be optimized by those skilled in the art in a known manner.

In still another aspect, the present invention relates to a method for inhibiting and/or preventing the differentiation of mature chondrocytes to hypertrophic chondrocytes comprising administrating to a subject in need thereof a therapeutically effective amount of Wnt6 or a derivative thereof.

Such differentiation into hypertrophic chondrocytes occurs in pathologies including osteoarthritis (OA), chondrocalcinosis, chondromalacia, cartilage defect, cartilage injury related to chronic inflammatory arthritis.

### Pharmaceutical Compositions

In another aspect, the present invention further relates to a pharmaceutical composition comprising Wnt6 or a derivative thereof, a nucleic acid molecule encoding for thereof or a vector comprising such nucleic acid, a population of chondrocytes obtainable according to a method described above or a population of host cells genetically engineered with said nucleic acid molecule or with said vector, eventually associated with a pharmaceutically acceptable vehicle.

The pharmaceutical composition may generally include one or more pharmaceutically acceptable and/or approved carriers, additives, antibiotics, preservatives, adjuvants, diluents and/or stabilizers. Such auxiliary substances can be water, saline, glycerol, ethanol, wetting or emulsifying agents, pH buffering substances, or the like. Suitable carriers are typically large, slowly metabolized molecules such as proteins, polysaccharides, polylactic acids, polyglycollic acids, polymeric amino acids, amino acid copolymers, lipid aggregates, or the like. This pharmaceutical composition can contain additional additives such as mannitol, dextran, sugar, glycine, lactose or polyvinylpyrrolidone or other additives such as antioxidants or inert gas, stabilizers or recombinant proteins (e.g. human serum albumin) suitable for in vivo administration.

As used herein, the term “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

In another aspect, the present invention relates to an endo-prosthesis for repairing lesions of the cartilage wherein said endo-prosthesis is coated with Wnt6 or a derivative thereof, a vector comprising a nucleic acid molecule encoding for Wnt6, or a host cell transformed with said nucleic acid molecule or said vector.

In one embodiment, said endo-prosthesis according to the invention can also be referred to as a chondral stent.

In another embodiment, the endo-prosthesis is further coated with at least another chondrogenic factor which is selected from the group consisting for example of FGF-2, FGF-5, FGF18, IGF-1, TGF-beta1-3, BMP-2, BMP-7, Shh, Sox9, PDGF and VEGF.

According to this aspect of the invention, Wnt6, or a vector comprising a nucleic acid molecule encoding for Wnt6, or a host cell transformed with said nucleic acid molecule or said vector can be placed within a biogel which coats the endo-prosthesis, or can be incorporated into the endo-prosthesis itself when it is made out of a material forming nanopores. Different methods for coating an endo-prosthesis are known in the field of “coated stents” or “drug-eluting stents” and can be applied for coating the endo-prosthesis of the invention. Such methods are disclosed, for example, in WO 97/037617 and WO 2005/016187.

The endo-prosthesis according to the invention can be used as an implant for repairing a lesion of the cartilage. The surgeon can implant the endo-prosthesis according to surgical procedures known in the art for treating lesions of the cartilage.

The invention will be further illustrated by the following figures and examples. However, these examples and figures should not be interpreted in any way as limiting the scope of the present invention.

### FIGURES

FIG. 1 shows the expression of the Wnt family members during the chondrogenic differentiation of primary human MSC. (A) Real-time RT-PCR analysis for chondrogenic markers on MSC cultured in BMP-2 conditioned medium on day 7 and 21 after chondrogenesis induction. (B and C) Real-time RT-PCR analysis for Wnt members on MSC cultured in BMP-2 conditioned medium from day 0 to day 21 after chondrogenesis induction. Results are expressed as the mean of two MSC samples in duplicates±standard deviation.

FIG. 2 shows the effect of Wnt-6 on the chondrogenic differentiation of MSC. (A) Semi-quantitative RT-PCR analysis for chondrogenic markers on MSC cultured in various conditioned media. MSC were the C3H10T1/2 murine cell line, bone marrow-derived primary murine (mMSC) and human MSC (hMSC). (B) Real-time RT-PCR analysis for chondrogenic markers on hMSC cultured in various media (Neg Ctrl: negative control, i.e. proliferative medium; TGFβ-3 containing medium; NIH or NIH-Wnt6 conditioned media). Results are expressed as the mean of three MSC samples in duplicates±standard deviation; *: p<0.01; **: p<0.001.

FIG. 3 shows the effect of Wnt-6 on the osteogenic differentiation of MSC. (A) Real-time RT-PCR analysis for osteogenic markers on MSC cultured in various media (Osteo Ctrl: osteogenic medium control; BMP-2 containing medium, NIH or NIH-Wnt6 conditioned media). Results are expressed as the mean of three MSC samples in duplicates±standard deviation; **: p<0.001. (B) Histochemical staining performed on one representative MSC sample out of 3 cultured as in (A): alizarin red S staining (upper panel) and alkaline phosphatase staining (lower panel).

FIG. 4 shows the effect of Wnt-6 on the adipogenic differentiation of MSC. (A) Real-time RT-PCR analysis for adipogenic markers on MSC cultured in various media (Neg Ctrl: negative control, i.e. proliferative medium; Osteo Ctrl: osteogenic positive control; NIH or NIH-Wnt6 conditioned media). Results are expressed as the mean of three MSC samples in duplicates±standard deviation; *: p<0.01; **: p<0.001. (B) Histochemical staining performed on one representative MSC sample out of 3 cultured as in (A): oil red 0 staining (right panel) and oil red O quantification after staining extraction and measure of the optical density at 492 nm (left panel).

FIG. 5 shows that Wnt-6 does not induce the expression of BMPs nor the canonical β-catenin pathway in MSC. MSC monolayers were treated with control, NIH or NIH-Wnt6 conditioned media. (A, B) The expression of BMPs was determined by semi-quantitative RT-PCR on day 1 (A) and 3 (B) of culture. (C) The expression of β-catenin was assessed at 48 h by Western-blotting. TGFβ-3 and LiCl were used as positive controls. (D) Phosphorylation of JNK was determined by Western-blotting. Cells were treated for 30 min using IL-1β as a positive control. The results are representative of at least three independent experiments.

FIG. 6 shows the role of Wnt-6 on the chondrogenic differentiation of primary murine MSC (DBA1) with the induction of markers specific for mature chondrocytes. (A, B, C) Real time RT-PCR analysis for chondrogenic markers on MSC cultured in micropellet in various conditioned media on day 21. (J0: day 0; BMP-2 containing medium; NIH or NIH-Wnt6 conditioned media; Agg: aggrecan; COMP: cartilage oligomeric protein; Col1: collagen 1 expressed by MSC and not by chondrocytes). Results are representative of at least three MSC samples in duplicates.

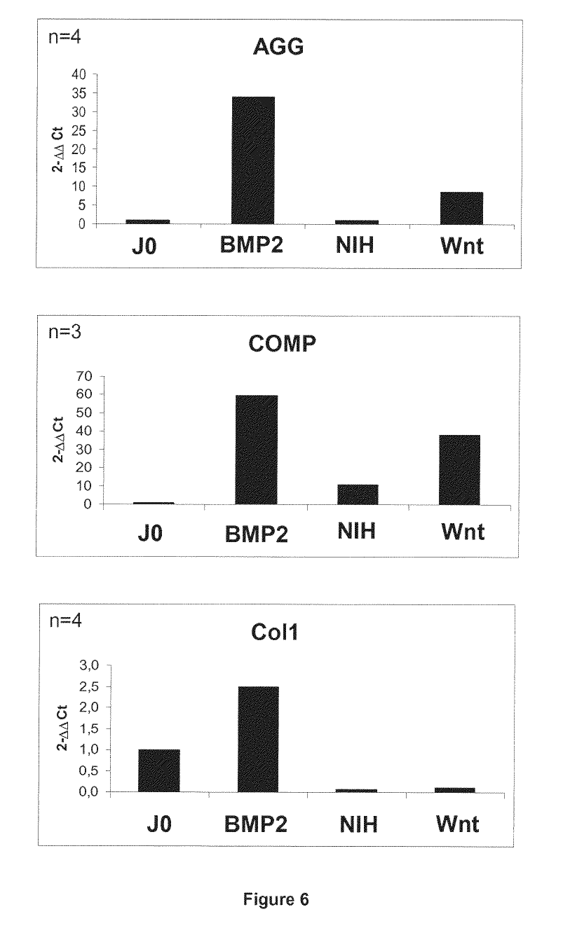

FIG. 7 shows the role of Wnt-6 on the hypertrophic chondrogenic differentiation of primary murine MSC with no induction of markers specific for hypertrophic chondrocytes (A, B, C) Real time RT-PCR analysis for markers of hypertrophic chondrogenic on MSC cultured in micropellet in various conditioned media on day 21. (J0: day 0; BMP-2 containing medium; NIH or NIH-Wnt6 conditioned media; Col10: collagen 10; PA: phosphatase alkaline; MMP13: metalloproteinase 13). Results are representative of at least three MSC samples in duplicates.

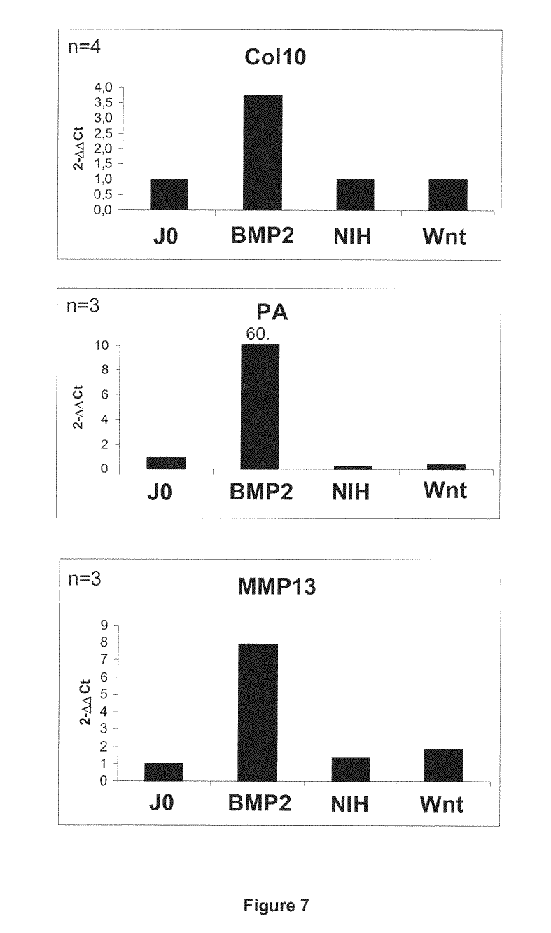

FIG. 8 shows the role of Wnt-6 on the osteogenic differentiation of primary murine MSC with no induction of markers specific for osteoblasts. (A, B, C) Real time RT-PCR analysis for markers of osteoblasts on MSC cultured in various conditioned media on day 21. (J0: day 0; Ost: osteogenic medium; BMP-2 containing osteogenic medium; NIH or NIH-Wnt6 osteogenic conditioned media; OC: osteocalcin; Col1: collagen 1; PA: phosphatase alkaline). Results are representative of at least two MSC samples in duplicates.

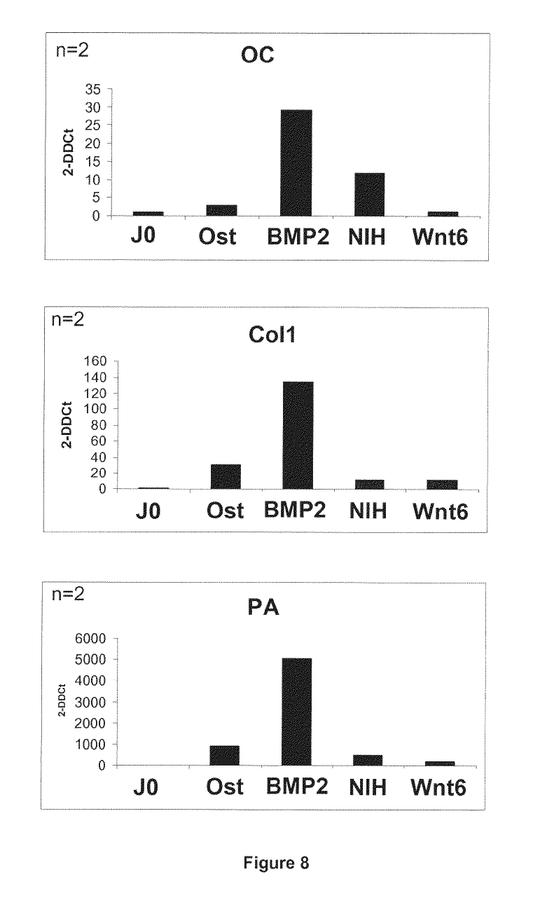

FIG. 9 shows the role of Wnt-6 on the adipogenic differentiation of primary murine MSC with no induction of markers specific for adipocytes. (A, B) Real time RT-PCR analysis for markers of adipocytes on MSC cultured in various conditioned media on day 21. (J0: day 0; Adipo: adipogenic medium NIH or NIH-Wnt6 adipogenic conditioned media; PPAR-g: peroxisome proliferator accelerated receptor-gamma; Fabp4: fatty acid binding protein 4). (C) Oil red O staining of lipid droplets. Results are representative of at least two MSC samples in duplicates.

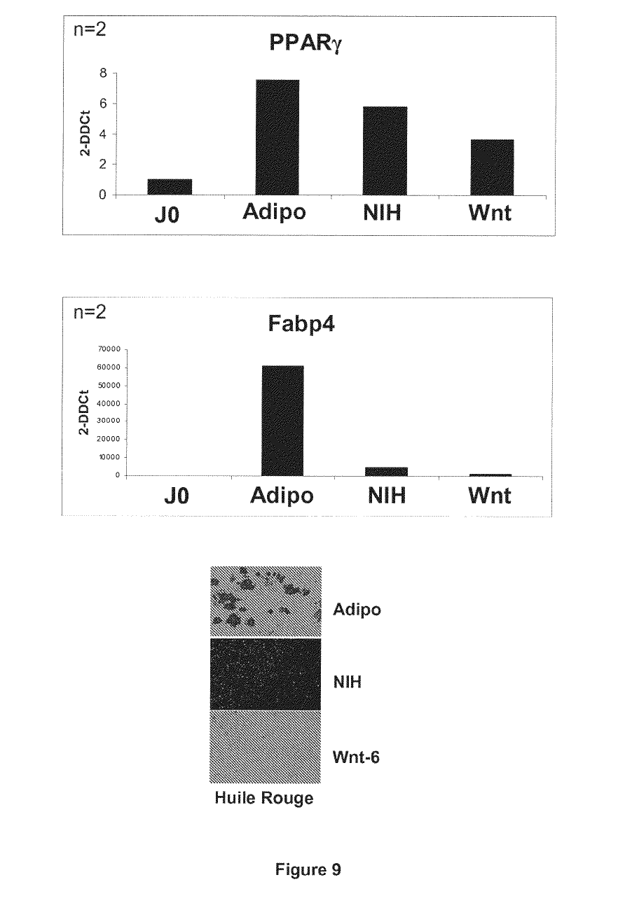

FIG. 10 shows the induction kinetics of chondrogenic differentiation induced by Wnt-6. (A, B, C,) Real time RT-PCR analysis for markers of chondrocytes on MSC cultured in micropellet in various conditioned media at different time points. (J0: day 0; Wnt: NIH-Wnt6 conditioned media; BMP2: BMP-2 conditioned medium; Agg: aggrecan; Col1: collagen 1; COMP: cartilage oligomeric protein). (D) Semi-quantitative RT-PCR for collagen 2B expression. Results are representative of at least two MSC samples in duplicates.

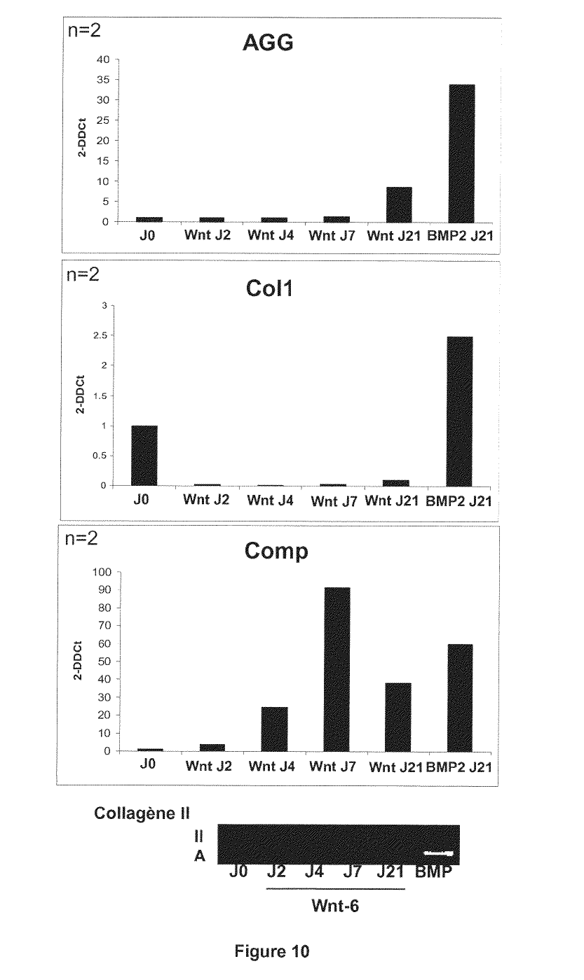

FIG. 11 shows the induction kinetics of hypertrophic chondrogenic differentiation induced by Wnt-6. (A, B, C) Real time RT-PCR analysis for markers of hypertrophic chondrocytes on MSC cultured in micropellet in various conditioned media at different time points. (J0: day 0; Wnt: NIH-Wnt6 conditioned media; BMP2: BMP-2 conditioned medium; Col10: collagen 10; MMP13: metalloproteinase 13; PA: phosphatase alkaline).

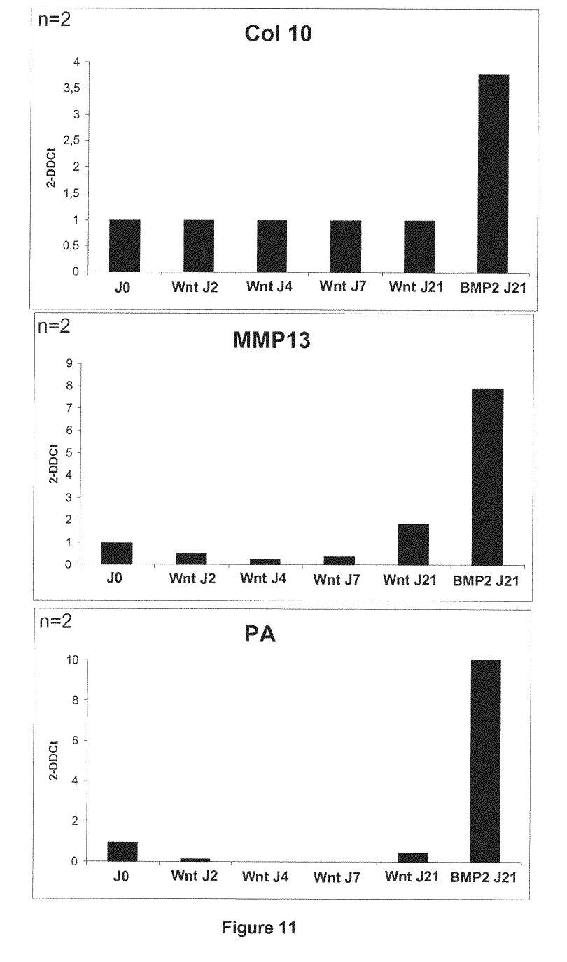

### EXAMPLE

Material & Methods

Cell culture: The NIH-3T3 (NIH) fibroblasts and C3H10T1/2 (C3) MSC line were cultured in Dulbecco's Modified Eagle medium (DMEM), supplemented with 10% foetal calf serum (FCS), 100 mM L-glutamine, 100 U/ml penicillin and 100 μg/ml streptomycin (Invitrogen, Cergy, France). The NIH-3T3 fibroblasts expressing murine Wnt6 (NIH-Wnt6) was kindly provided by S. Vainio (Itaranta et al., 2002). The human primary MSC (hMSC) were isolated from bone marrow as previously described (Djouad et al., 2005) and used at passages 2 to 3. The murine primary MSC (mMSC) were obtained after flushing the bone marrow from the femurs and tibias of DBA/1 mice and culture of mononuclear cells in DMEM containing 10% FCS. After passage 5, the adherent cell population was characterized by flow cytometry to homogeneously express Sca1, CD44, CD73 and not CD45, CD11b and differentiation potential.

Differentiation assays: Chondrogenic differentiation of MSC was induced by culture in micropellet in presence of incomplete chondrogenic medium, or complete chondrogenic medium containing either 10 ng/ml TGFbeta3 or BMP-2 conditioned medium as previously described (Djouad et al., 2005). Briefly, MSC (2.5×10⁵ cells) were pelleted by centrifugation in 15 ml conic tubes and cultured for 21 days with medium changes every other day. Osteogenic and adipogenic differentiations were induced by culture in monolayers for 21 days in specific media (Djouad et al., 2005). Wnt6 conditioned or control media were obtained after 48 h incubation of DMEM medium on confluent NIH-Wnt6 or NIH cells. The various components of each specific differentiation medium were added to the conditioned media just before incubation with MSC.

Histological staining: Pellets were fixed in 4% formaldehyde, embedded in paraffin and 5 μm-thick sections were prepared. Presence of proteoglycans was visualized by incubation with a 0.1% Safranin O solution for 5 min. For evaluation of mineralized matrix, cells were fixed with ethanol 95% for 30 min, rinsed with PBS and stained with Alizarin red S (Sigma, l'Isle d'Abeau, France) for 5 min, followed by a rapid wash with acetone:methanol solution (1:1). To evaluate the presence of neutral lipids, cells were fixed with 3% glutaraldehyde for 1 h, stained with oil red 0 solution for 2 h and washed with 60% isopropanol. Immunohistochemical analysis was performed for type II collagen and aggrecan, using the primary antibodies from Interchim (Montluçon, France) and Chemicon (Hampshire, United Kingdom), respectively. Immunostaining was then performed using the “Ultravision Mouse Tissue Detection System” kit from Lab Vision Corporation (Fremont, Calif., USA).

RNA preparation: MSC cultured in monolayer or in micropellets (10-15) were washed with phosphate buffered saline (PBS) and treated with lysis buffer. Total RNA was extracted using the RNeasy Kit according to the recommendations of the supplier (Quiagen S. A., Courtaboeuf, France).

TaqMan real time RT-PCR: TaqMan low density arrays (TLDA; microfluidic cards, Applied Biosystems, Courtaboeuf, France) were used in a two step RT-PCR process. First strand cDNA was synthesized from 3 μg total RNA using the High Capacity cDNA Archive Kit (Applied Biosystems). Real time PCR reactions were then carried out with predesigned fluorogenic TaqMan probes and primers using 384 well microfluidic cards as previously described (Djouad et al., 2005). The complete list of transcripts is available in (Djouad et al., 2005). Data were analysed using the threshold cycle (Ct) relative quantification method. Content of cDNA samples was normalized by subtracting the number of copies of the endogenous GAPDH reference gene to the target gene (ΔCt=Ct of target gene−Ct of GAPDH). The results are expressed as the mean of 2^{-ΔCt}±the standard error of the mean (SEM).

Semi-quantitative RT-PCR: RT-PCR was performed on 1 μg of RNA using the GeneAmp® RNA PCR Core Kit (Applied Biosystems). The primers for murine collagen II, aggrecan, glyceraldehyde-3-phosphate dehydrogenase (GAPDH) and PCR conditions were already described (Noel et al., 2004). PCR products were electrophoresed in 1% agarose gel, stained with ethidium bromide and visualized by ultraviolet transillumination.

Western blot: Cells were lysed in lysis buffer containing 10 mM Tris-HCl, pH 7,4; 30 mM NaPPi; 1% triton X100; 50 mM NaCl; 1 mM EDTA; 20 mM β-glycerophosphate; 1 mM EGTA; 100 μM Na₃VO₄; Protease inhibitor cocktail; 50 nM acid okadaic; 100 μM phenylmethanesulfonyl fluoride (PMSF) and 1 mM DTT (Sigma, l'Isle d'Abeau, France). Lysis was allowed to proceed at 4° C. for 30 min before elimination of nuclei by centrifugation (15.700 g at 4° C. for 10 min). The protein concentration was determined using the bicinchoninic acid (BCA) kit according to manufacturer's recommendations (Sigma). Cell extracts were then mixed with 1 volume of 2× Laemmli electrophoresis loading buffer (125 mM Tris-HCl, pH 6.8; 2% SDS; 10% glycerol; 720 mM β-mercaptoethanol; 0.125% bromophenol blue) and boiled for 3 min. Proteins (10 μg) were electrophoresed on a 10% SDS-polyacrylamide gel and transferred to a polyvinylidene difluoride (PVDF) membrane (Biorad, Marne la coquette, France). Immunodetection was performed by incubating the primary antibodies overnight at 4° C. The anti-β-catenin and anti-JNK, anti-JNK phosphorylated, anti-PKC were respectively from Upstate, New York and Cell Signaling, Ozyme. The horseradish peroxidase-coupled anti-mouse or goat anti-rabbit secondary antibodies (Jackson Immuno Research Laboratories, West Grove, Pa.) were incubated for 1 h at room temperature. The signal was detected with the Western Lightning Chemiluminescence Reagent Plus (PerkinElmer LAS, Inc).

Results

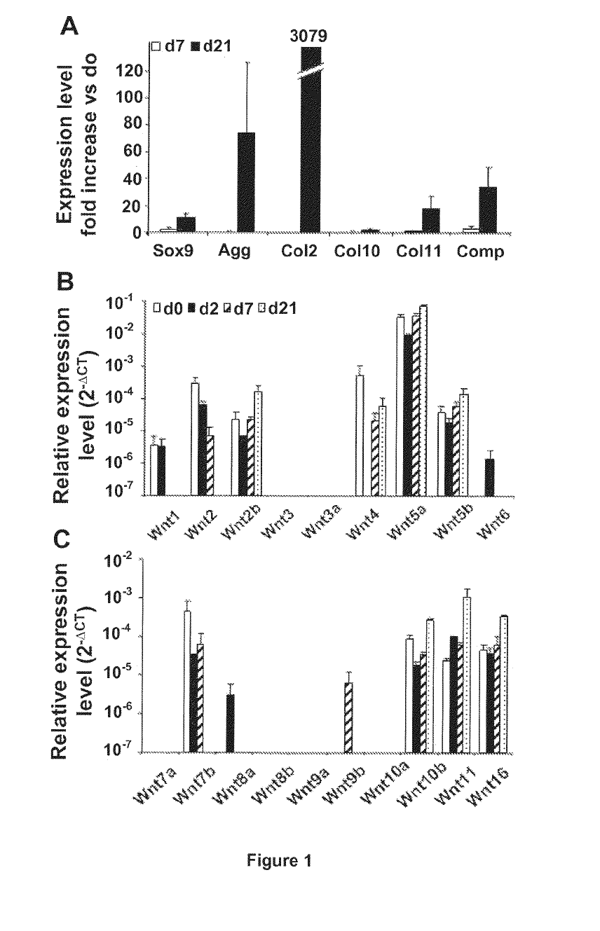

Expression kinetics of Wnts and their receptors during chondrogenesis: Using DNA microarrays, we previously showed that various members of the Wnt family were differentially expressed in MSC after 21 days of in vitro induced chondrogenesis. Here, we investigated on a quantitative basis the expression kinetics of the majority of the Wnts, receptors and antagonists during the chondrogenic process. The differentiation of hMSC was induced by culture in micropellets in presence of BMP-2 and confirmed by the detection of the major chondrocytic markers on day 21 (FIG. 1A). In parallel, 10 out the 19 studied Wnts were expressed in MSC. Among them, 6 (Wnt2b, Wnt5a, Wnt5b, Wnt10b, Wnt11, Wnt16) were up-regulated on day 21 and 4 were absent (Wnt1, Wnt2, Wnt7b) or down-regulated (Wnt4) on day 21 (FIG. 1B, C). Interestingly, 3 members were not expressed in MSC and transitorily up-regulated on day 2 (Wnt6 and Wnt8a) or day 7 (Wnt9b) of chondrogenesis. Indeed, the temporary and early expression of Wnt6 in this process together with its expression in zones of intervertebral disk and joint formation during embryogenesis made this molecule a potential chondroinductive candidate.

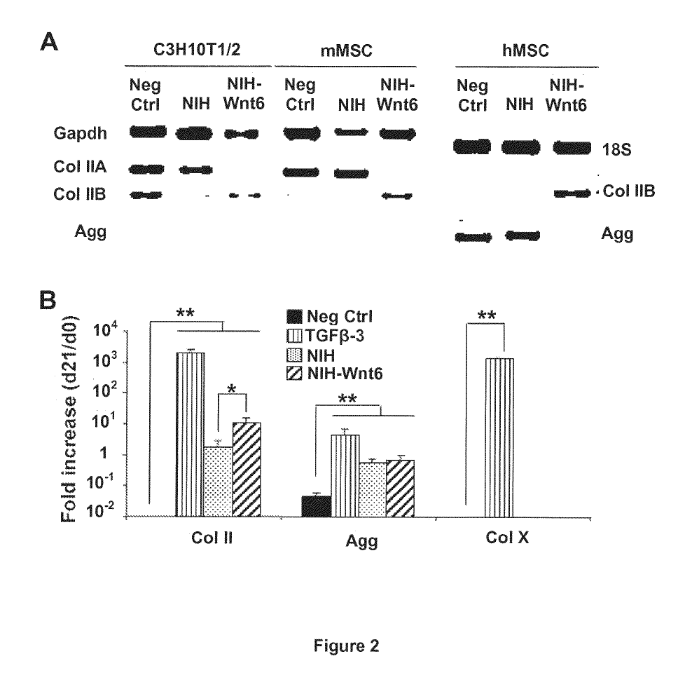

Role of Wnt6 in inducing MSC chondrogenic differentiation: In absence of the recombinant protein, we used a fibroblastic cell line expressing the murine form of Wnt6 which shares 97% homology at the nucleic acid level with its human counterpart. First, we investigated whether a conditioned medium containing the secreted murine Wnt6 may induce the differentiation of MSC from various sources, the murine C3 MSC line, mMSC or hMSC. As shown in FIG. 2A, the conditioned medium from NIH-Wnt6 cells was able to up-regulate the expression of the mRNAs coding for the chondrocytic markers, the collagen type IIB and aggrecan. On the contrary, absence or low expression of these markers was observed when MSC were incubated in presence of conditioned medium from NIH cells or proliferative medium (FIG. 2A). Second, we relied on real time RT-PCR to precisely quantify the increase of chondrocyte specific transcripts in a similar differentiation experiment using hMSC. Again, the use of Wnt6-containing conditioned medium up-regulated the expression of levels of collagen type II and aggrecan but not collagen type X suggesting that hypertrophic differentiation did not occur (FIG. 2B). Altogether, the results suggest that mWnt6 can induce the in vitro differentiation of hMSC towards chondrocytes.

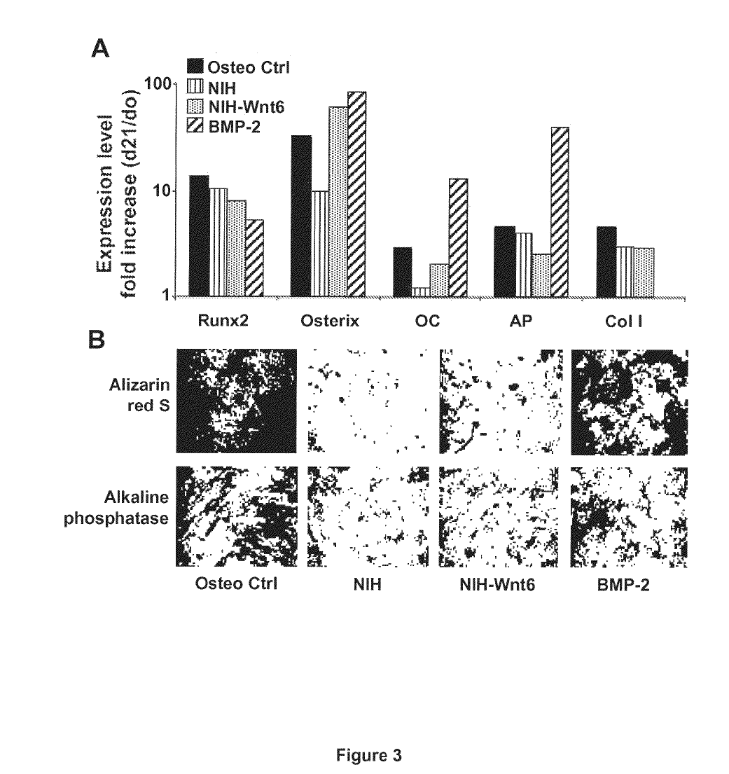

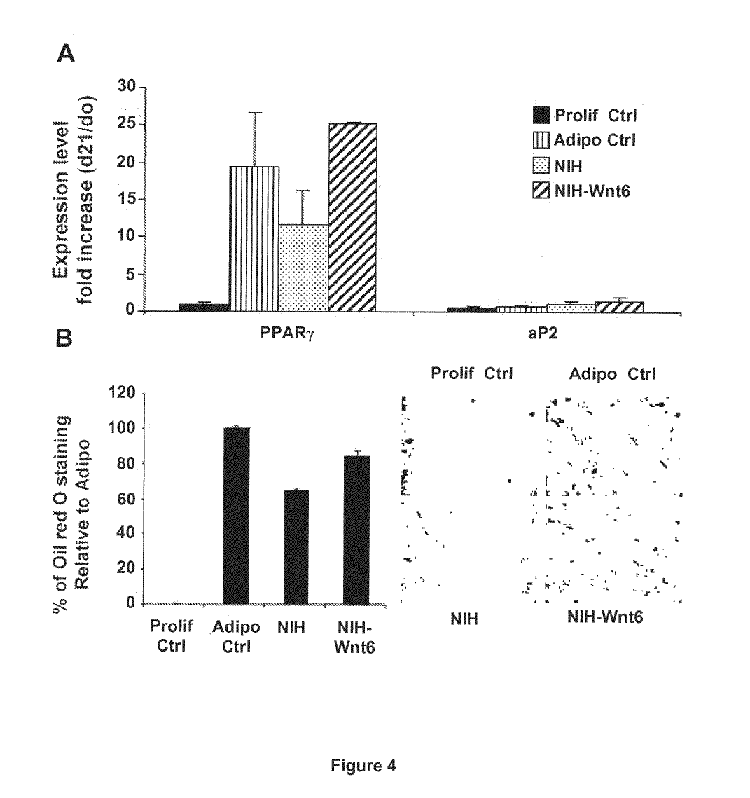

Role of Wnt6 in osteogenic and adipogenic differentiations: Specificity of the inductive effect was then tested on the differentiation of MSC towards osteoblastic and adipocytic lineages. The increase of mRNA levels of bone markers was not significantly different for MSC cultured with NIH-Wnt6 conditioned medium, NIH conditioned medium or the osteoblastic control medium (FIG. 3A). However, a higher up-regulation of osterix, alkaline phosphatase and osteocalcin was observed when MSC were incubated in presence of BMP-2. This was not observed at the protein level since alkaline phosphatase activity was reduced in all samples as compared to the osteoblastic control (FIG. 3B). Similarly, the capacity to mineralize the extracellular matrix was reduced when MSC were cultured in presence of NIH or NIH-Wnt6 conditioned media, suggesting the secretion of an inhibitory factor by the NIH fibroblasts and no influence of Wnt6 on the differentiation potential of MSC when cultured under osteoblastic conditions. In adipogenic culture conditions, hMSC differentiated into adipocytes as demonstrated by increase of the PPARγ, LPL adipogenic markers and the formation of lipid droplets (FIG. 4A, B). hMSC incubated with NIH or NIH-Wnt6 conditioned media exhibited similar capacity to form lipid droplets and no significant difference in the expression level of adipogenic markers, suggesting no or little impact of Wnt6 on the adipogenic differentiation of MSC.

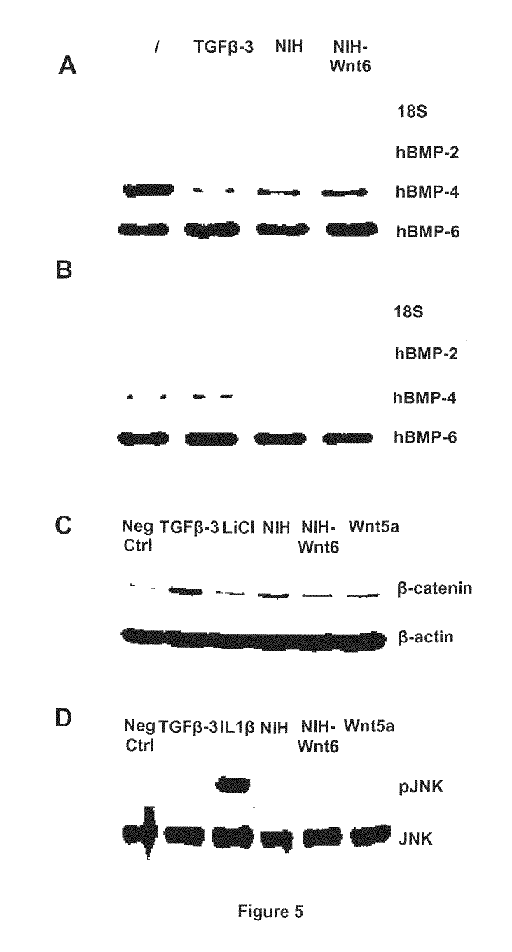

Direct or indirect role of Wnt6: The use of a conditioned medium does not allow to exclude the possibility that Wnt6 may act through the induction of another soluble mediator which may be the real inductive factor. To determine whether the contribution of Wnt6 was direct or indirect, we evaluated the expression of a number of BMP transcripts by hMSC cultured in presence of the conditioned media. No expression of BMP-3, BMP-7, BMP-9 or BMP-15 could be detected at any time points. On the contrary, a low up-regulation of BMP-2 in parallel with strong down-regulation of BMP-4 and steady-state levels of BMP-6 were observed when MSC were cultured in presence of Wnt6-conditioned medium on day 3 of culture (FIG. 5A, 5B). These results suggest that osteo-chondro-inductive BMP members may be regulated by Wnt6 in culture but it likely does not account for the induction of chondrogenesis in vitro.

Wnt signalling pathway activation: An experimental evidence for a direct role of Wnt6 on chondrogenesis is to demonstrate the activation of the signalling pathways shared by sub-classes of Wnt members. Indeed, we first investigated the activation of the β-catenin dependent canonical pathway after culture of hMSC in monolayers for 2 days in presence of TGFβ3, LiCL or NIH and NIH-Wnt6 conditioned media. As control, TGFβ3 addition increased the protein level of β-catenin, reflecting the accumulation of the protein in the cells (FIG. 5C). In the other extracts, no up-regulation of β-catenin levels was detected, suggesting that Wnt6-containing medium is unable to activate the canonical Wnt pathway in these conditions. Second, we determined whether Wnt6-containing medium may induce the JNK signalling pathway. We used IL-1β as a control and detected the phosphorylated form of JNK in corresponding cell extracts while phospho-JNK was not expressed in cell extracts prepared from hMSC incubated in presence of Wnt5a, NIH or NIH-Wnt6 conditioned media (FIG. 5D). The levels of total JNK were similar in all cell extracts. Altogether, the results reflect that Wnt6-containing medium induces chondrogenesis independently of the β-catenin or the JNK signalling pathway.

### REFERENCES

Chen, Y., Whetstone, H. C., Youn, A., Nadesan, P., Chow, E. C., Lin, A. C., and Alman, B. A. (2007) J Biol Chem 282(1), 526-533.
Chung Y, Klimanskaya I, Becker S, Li T, Maserati M, Lu S J, Zdravkovic T, Ilic D, Genbacev O, Fisher S, Krtolica A, Lanza R, Cell Stem Cell. 2008 Feb. 7; 2(2):113-7.
Church V, Nohno T, Linker C, Marcelle C, Francis-West P. “Wnt regulation of chondrocyte differentiation” J Cell Sci. 2002 Dec. 15; 115(Pt 24):4809-18.
Clevers, H. (2006) Cell 127(3), 469-480.
Day, T. F., Guo, X., Garrett-Beal, L., and Yang, Y. (2005) Dev Cell 8(5), 739-750
Dellavalle A et al., “Pericytes of human skeletal muscle are myogenic precursors distinct from satellite cells”, Nat. Cell Biol., 2007, 9: 255-267.
Djouad, F., Bony, C., Haupl, T., Uze, G., Lahlou, N., Louis-Plence, P., Apparailly, F.,
Canovas, F., Reme, T., Sany, J., Jorgensen, C., and Noel, D. (2005) Arthritis Res Ther 7(6), R1304-1315
Dominici, M., Le Blanc, K., Mueller, I., Slaper-Cortenbach, I., Marini, F., Krause, D., Deans, R., Keating, A., Prockop, D., and Horwitz, E. (2006) Cytotherapy 8(4), 315-317.
D'ippolito G et al., “Marrow-isolated adult multilineage inducible (MIAMI) cells, a unique population of postnatal young and old human cells with extensive expansion and differentiation potential”, J. Cell Sci., 2004, 117 (Pt 4): 2971-2981.
Enomoto-Iwamoto, M., Kitagaki, J., Koyama, E., Tamamura, Y., Wu, C., Kanatani, N., Koike, T., Okada, H., Komori, T., Yoneda, T., Church, V., Francis-West, P. H., Kurisu, K., Nohno, T., Pacifici, M., and Iwamoto, M. (2002) Dev Biol 251(1), 142-156.
Etheridge, S. L., Spencer, G. J., Heath, D. J., and Genever, P. G. (2004) Stem Cells 22(5), 849-860.
Fischer, L., Boland, G., and Tuan, R. S. (2002) J Biol Chem 277(34), 30870-30878.
Gavin, B. J., McMahon, J. and McMahon, A. (1990) Genes Dev 4(12B), 2319-2332.
Gordon, M. D., and Nusse, R. (2006) J Biol Chem 281(32), 22429-22433.
Hill, T. P., Spater, D., Taketo, M. M., Birchmeier, W., and Hartmann, C. (2005) Dev Cell 8(5), 727-738.
Itaranta, P., Lin, Y., Perasaari, J., Roel, G., Destree, O., and Vainio, S. (2002) Genesis 32(4), 259-268.
Jin, E. J., Park, J. H., Lee, S. Y., Chun, J. S., Bang, O. S., and Kang, S. S. (2006) Int J Biochem Cell Biol 38(2), 183-195.
Jin, E. J., Lee, S. Y., Choi, Y. A., Jung, J. C., Bang, O. S., and Kang, S. S. (2006) Mol Cells 22(3), 353-359.
Kikuchi, A., Yamamoto, H., and Kishida, S. (2007) Cell Signal 19(4), 659-671.
Krishnan, V., Bryant, H, and Macdougald, O (2006) J Clin Invest 116(5), 1202-1209.
Kögler G. et al., “A new human somatic stem cell from placental cord blood with intrinsic pluripotent differentiation potential”, J. Exp. Med., 2004, 200(2): 123-135.
Linker, C., Lesbros, C., Gros, J., Burrus, L. W., Rawls, A., and Marcelle, C. (2005) Development 132(17), 3895-3905.
Moll, F., Millet, C., Noel, D., Orsetti, B., Bardin, A., Katsaros, D., Jorgensen, C., Garcia, M., Theillet, C., Pujol, P., and Francois, V. (2006) Faseb J 20(2), 240-250.
Noel, D., Gazit, D., Bouquet, C., Apparailly, F., Bony, C., Plence, P., Millet, V., Turgeman, G., Perricaudet, M., Sany, J., and Jorgensen, C. (2004) Stem Cells 22(1), 74-85.
Revazova E S, Turovets N A, Kochetkova O D, Agapova L S, Sebastian J L, Pryzhkova M V, Smolnikova V I, Kuzmichev L N, Janus J D, “HLA homozygous stem cell lines derived from human parthenogenetic blastocysts”, Cloning Stem Cells. 2008 March; 10(1):11-24 Reyes M et al., “ ”, J. Clin. Invest., 2002, 109(3): 337-346.
Ryu, J. H., and Chun, J. S. (2006) J Biol Chem 281(31), 22039-22047.
Sampaolesi M et al., “Mesangioblast stem cells ameliorate muscle function in dystrophic dogs”, Nature, 2006, 444(7119): 574-579.
Schmidt, C., Stoeckelhuber, M., McKinnell, I., Putz, R., Christ, B., and Patel, K. (2004) Dev Biol 271(1), 198-209.
Schubert, F., Mootoosamy, R., Walters, E., Graham, A., Tumiotto, L., Munsterberg A., Lumsden, A., and Dietrich, S. (2002) Mech Dev 114(1-2), 143-148.
Shimizu, H., Julius, M. A., Giarre, M., Zheng, Z., Brown, A. M., and Kitajewski, J. (1997) Cell Growth Differ 8(12), 1349-1358.
Spater, D., Hill, T. P., Gruber, M., and Hartmann, C. (2006) Eur Cell Mater 12, 71-80.
Takahashi K, Tanabe K, Ohnuki M, Narita M, Ichisaka T, Tomoda K, Yamanaka S. Cell. 2007 Nov. 30; 131(5):861-72.
Yu J, Vodyanik M A, Smuga-Otto, K, et al. Science 318, 1917-1920 (2007).

1. A method for obtaining a population of chondrocytes, said method comprising a step of culturing pluripotent or multipotent stem cells with a culture medium comprising Wnt6 or a derivative thereof.

2. (canceled)

3. The method according to claim 1, wherein the multipotent stem cells are multipotent mesenchymal stromal cells (MSCs).

4. The method according to claim 1, wherein the culture medium further comprises at least another chondrogenic factor, which is selected from the group consisting of FGF-2, FGF-5, FGF-18, IGF-1, TGF-beta1-3, BMP-2, BMP-7, Shh, Sox9, PDGF and VEGF.

5. The method according to claim 1, wherein the step of culturing is carried out for about 4 days.

6. The method according to claim 1, wherein Wnt6 is immobilized on a solid phase or delivered by a scaffold.

7. A population of chondrocytes obtained by the process of culturing pluripotent or multipotent stem cells with a culture medium comprising Wnt6 or a derivative thereof.

8. A population of pluripotent or multipotent stem cells which have been transformed with a nucleic acid molecule encoding for Wnt6 or a derivative thereof or a vector comprising such nucleic acid.

9. A pharmaceutical composition comprising Wnt6 or a derivative thereof, a nucleic acid molecule encoding for thereof or a vector comprising such nucleic acid, and a population of host cells which is either a population of chondrocytes obtained by the process of culturing pluripotent or multipotent stem cells with a culture medium comprising Wnt6 or a derivative thereof, or a population of pluripotent or multipotent stem cells which have been transformed with a nucleic acid molecule encoding for Wnt6 or a derivative thereof or a vector comprising such nucleic acid.

10. The pharmaceutical composition according to claim 9, wherein the population of host cells transformed is the population of pluripotent or multipotent stem cells.

11. A method for regenerating cartilage and/or treating an osteo-articular pathology in a subject, comprising the step of administering to said subject a therapeutic dose of Wnt6 polypeptide.

12. The method according to claim 10, wherein said osteo-articular pathology is selected from the group consisting of degenerative joint disease as osteoarthritis (OA) and inflammatory joint diseases as rheumatoid arthritis (RA) and ankylo sing arthritis (AS) and traumatic pathologies.

13. A method for treating and/or preventing an osteo-articular pathology in a subject comprising a step of administering to a subject in need thereof a therapeutically effective amount of Wnt6 or a derivative thereof.

14. A method for inhibiting and/or preventing the differentiation of mature chondrocytes to hypertrophic chondrocytes comprising the step of administering to a subject in need thereof a therapeutically effective amount of Wnt6 or a derivative thereof.

15. An endo-prosthesis for repairing lesions of the cartilage wherein said endo-prosthesis is coated with Wnt6 or a derivative thereof, a vector comprising a nucleic acid molecule encoding for Wnt6, or a host cell transformed with said nucleic acid molecule or said vector.
